(19) United States
(12) Patent Application Publication
(10) Pub. No.: US 2010/0112681 A1
(43) Pub. Date: May 6, 2010

(54) MICROCHIP FLUID CONTROL MECHANISM

(75) Inventors: Minoru Asogawa, Tokyo (JP); Hisashi Hagiwara, Kanagawa (JP); Tohru Hiramatsu, Nagano (JP)

Correspondence Address: YOUNG & THOMPSON, 209 Madison Street, Suite 500, Alexandria, VA, 22314, US

(73) Assignees: NEC CORPORATION, TOKYO (JP); AIDA ENGINEERING, LTD., KANAGAWA (JP)

(21) Appl. No.: 12/530,377

(22) PCT Filed: Mar. 4, 2008
(86) PCT No.: PCT/JP2008/054243

§ 371 Date: Sep. 8, 2009

(30) Foreign Application Priority Data
Mar. 5, 2007 (JP) 2007-054041

(51) Int. Cl. G01N 35/08; G01N 37/00; G01N 33/50; B81B 7/04

(52) U.S. Cl. 435/288.5; 422/102; 422/101; 422/68.1

(57) ABSTRACT

Compressed gas is supplied from above to a specimen vessel having an open top. Transfer channels to reaction vessels are provided under a microchip and transfer channels extending from the reaction vessels are provided on the microchip. Transfer element is provided outside the microchip so as to supply the compressed gas from a member (cover) holding the microchip.

### TECHNICAL FIELD

This invention relates to a microchip fluid control mechanism and, in particular, to a micro-analysis chip used for reacting, mixing, separating or analyzing chemical specimens or for analyzing genes, and having a plurality of reaction vessels and specimen vessels, the reaction vessels and the specimen vessels being connected through microchannels.

### BACKGROUND ART

As described in “Micromachine Technology for Biochemical and Microchemical Analysis Systems” by SHOJI Shuichi (Non-Patent Document 1), and Japanese Laid-Open Patent Publication No. 2002-214241 (Patent Document 2), numerous studies have recently been made on gene analyses performed by reacting samples or liquid specimens on a micro reactor, a micro array, or a single microchip called “Lab-on-a-chip”. Studies also have been made on mechanisms for sequentially transferring extremely small volumes of liquid specimens and on controlling mechanisms.

Non-Patent Document 1 discloses in chapter “2. μTAS using micromachine elements” a structure formed on a single base and consisting of “a specimen introduction mechanism, a pump for controlling flows of carrier solution and sample, a mixer-reactor for mixing/reacting the same with a reagent, a component separation portion, and a sensor portion”. This Non-Patent Document 1 also describes “However, there are only few practical applications and experiences of this technology, and micro fluid control elements such as micro valves and micro pumps are important subjects to be studied for practical applications”.

Non-Patent Document 1 further discloses a structure in which a large number of complicated transfer means including micro pumps and specimen injectors are mounted on a single base.

Patent Document 2 mentioned above describes, “The micro pump 30 is incorporated in the channels 21, 23” (see paragraph [0039]), and transfer means is provided in a microchip.

Another conventional technology is disclosed in Japanese Laid-Open Patent Publication No. 2004-226207 (Patent Document 3). Patent Document 3 discloses a transfer mechanism using a diaphragm. Specifically, the transfer mechanism has, in addition to a partition having a possible elasticity, a diaphragm member in contact with the external face of the partition and an incompressible medium for driving the diaphragm member. According to Patent Document 3, the variation in volume of a closed container for “the incompressible medium” is precisely controlled, so that the diaphragm member is driven by the variation in volume and the liquid flow rate is controlled.

### DISCLOSURE OF THE INVENTION

### Problems to be Solved by the Invention

However, according to the conventional technologies described in Non-Patent Document 1 and Patent Document 2, specimen transfer means is provided within or on a microchip, and hence careful cleaning processes must be performed for preventing cross contamination when gene analyses are conducted consecutively. Furthermore, the microchip is increased in size and hence in cost. It has been considered that disposable microchips are desirable for preventing cross contamination.

According to the conventional technology disclosed in Patent Document 3, the use of an incompressible medium is indispensable and the use of a compressible medium is not possible.

This invention has been made in view of the problems entrained by the conventional technologies as described above, and it is an object of the invention to provide a microchip fluid control mechanism in which transfer means is provided independently from a microchip so that the use of unsophisticated, inexpensive and disposable microchips is made possible, and which makes it possible for devices to realize reduction in size and weight, increase of operating speed, reduction of power consumption, simplification of circuit and device configuration, cost reduction, and improvement in reliability and operability.

### Means for Solving the Problems

In order to achieve the object above, this invention provides a microchip fluid control mechanism having a plurality of specimen vessels each having an open top for receiving specimens, and a plurality of reaction vessels for mixing and reacting the specimens, the specimen vessels and the reaction vessels being mutually linked through a channel to sequentially transfer the specimens by a pressurizing unit so that the specimens are subjected to predetermined processing. The microchip fluid control mechanism is characterized in that a transfer channel from the specimen vessels and transfer channels to the reaction vessels are provided under the specimen vessels and the reaction vessels.

### Effects of the Invention

According to this invention, the need of a valve mechanism conventionally provided in microchips is eliminated to simplify the passage structure, whereby disposable and inexpensive microchip can be provided.

### BRIEF DESCRIPTION OF THE DRAWINGS

FIG. 1 is a cross-sectional perspective view showing a configuration of a microchip transfer mechanism according to a first embodiment of this invention;

FIG. 2 is a cross-sectional view showing a configuration of the microchip transfer mechanism according to the first embodiment of this invention;

FIG. 3 is a cross-sectional perspective view showing an initial state of the microchip according to the first embodiment of this invention;

FIG. 4 is a cross-sectional perspective view showing an operating state of the microchip according to the first embodiment of this invention;

FIG. 5 is a cross-sectional perspective view showing an operating state of the microchip according to the first embodiment of this invention;

FIG. 6 is a cross-sectional perspective view showing an operating state of the microchip according to the first embodiment of this invention;

FIG. 7 is a cross-sectional perspective view showing an operating state of the microchip according to the first embodiment of this invention;

FIG. 8 is a cross-sectional perspective view showing an operating state of the microchip according to the first embodiment of this invention;

FIG. 9 is a cross-sectional perspective view showing an operating state of the microchip according to the first embodiment of this invention;

FIG. 10 is a cross-sectional perspective view showing an operating state of the microchip according to the first embodiment of this invention;

FIG. 11 is a cross-sectional perspective view showing an operating state of the microchip according to the first embodiment of this invention;

FIG. 12 is a perspective view showing another embodiment of this invention;

FIG. 13 is a perspective view showing another embodiment of this invention;

FIG. 14 is a perspective view showing another embodiment of this invention;

FIG. 15 is a cross-sectional view showing an operating state according to another embodiment of this invention;

FIG. 16 is a cross-sectional view showing an operating state according to another embodiment of this invention; and

FIG. 17 is a flowchart showing operating states of the microchip according to the first embodiment of this invention.

### BEST MODE FOR CARRYING OUT THE INVENTION

Firstly, a first embodiment of this invention will be described in detail.

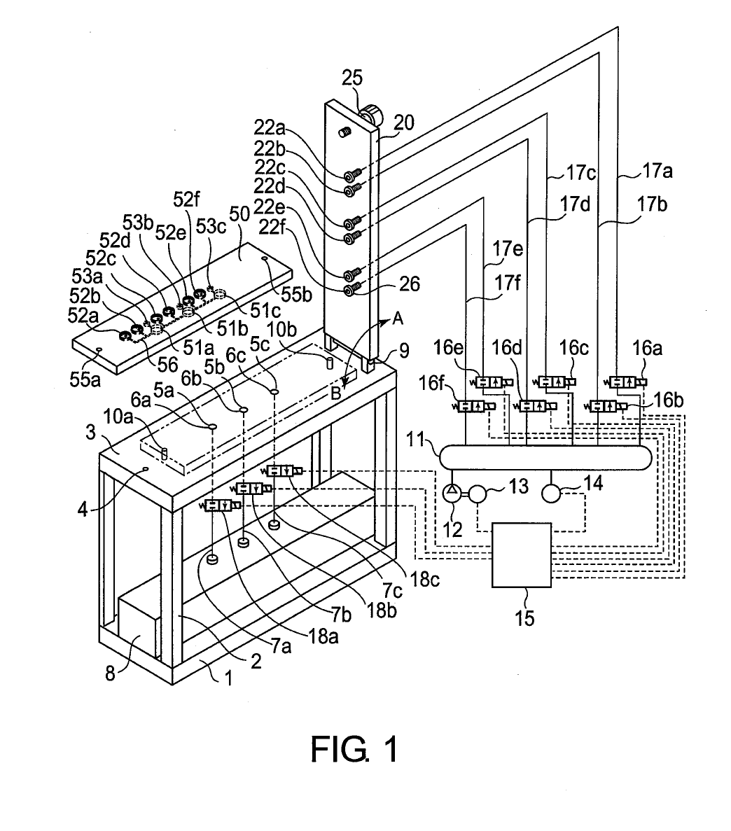

FIG. 1 is a cross-sectional perspective view showing a configuration of a device for reacting chemical specimens by using a microchip according to the first embodiment of the invention.

A table 3 is provided on a machine casing 1 by means of pillars 2, and the table 3 is provided with discharging holes 5a, 5b, 5c the peripheries of which are sealed with O-rings 6a, 6b, 6c, and tubes 7a, 7b, 7c. The discharging holes 5a, 5b, 5c are connected to a waste vessel 8 disposed on the machine casing 1, through discharging electromagnetic valves 18a, 18b, 18c. Pins 10a, 10b are provided so as to project from the upper surface of the table 3, for guiding a microchip 50 to a predetermined position. Further, a cover 20 is provided on the table 3 turnably in directions A and B by means of a hinge 9. The cover 20 has a fastening screw 25 and pressurizing holes 22a, 22b, 22c, 22d, 22e, 22f the peripheries of which are sealed with O-rings 26 and passing through the cover. Further, a threaded hole 4 is formed at an end of the table 3 at a position corresponding to the fastening screw 25.

On the other hand, the microchip 50 has a plate-like shape and is provided with reaction vessels 51a, 51b, 51c for mixing a plurality of specimens, and specimen vessels 52a, 52b, 52c, 52d, 52e, 52f for containing the specimens. Discharging holes 53a, 53b, 53c are linked to the reaction vessels 51a, 51b, 51c through a channel 56 for discharging the specimens having overflowed from the reaction vessels 51a, 51b, 51c. Pin holes 55a, 55b are opened at the opposite ends of the microchip 50 to guide the microchip to the position where it is mounted on the table 3.

The pressurizing holes 22a, 22b, 22c, 22d, 22e, 22f formed to pass through the cover 20 are connected to secondary sides of pressurizing electromagnetic valves 16a, 16b, 16c, 16d, 16e, 16f through tubes 17c, 17d, 17e, 17f. Primary sides of the pressurizing electromagnetic valves 16a, 16b, 16c, 16d, 16e, 16f are connected to an accumulator 11. The accumulator 11 is connected to a pump 12 driven by a motor 13, and a pressure sensor 14 for detecting internal pressure.

A controller 15 for executing a preinstalled program is connected to the pressurizing electromagnetic valves 16a, 16b, 16c, 16d, 16e, 16f and the discharging electromagnetic valves 18a, 18b, 18c such that operations thereof can be controlled. Further, the controller 15 is connected to the motor 13 for driving the pump 12 such that the pressure within the accumulator 11 can be controlled to a predetermined value, and to a pressure sensor 14 for detecting and feeding back the pressure within the accumulator 11. The configuration as described above allows the pressure within the accumulator 11 to be held constantly at a predetermined pressure according to a command from the controller 15.

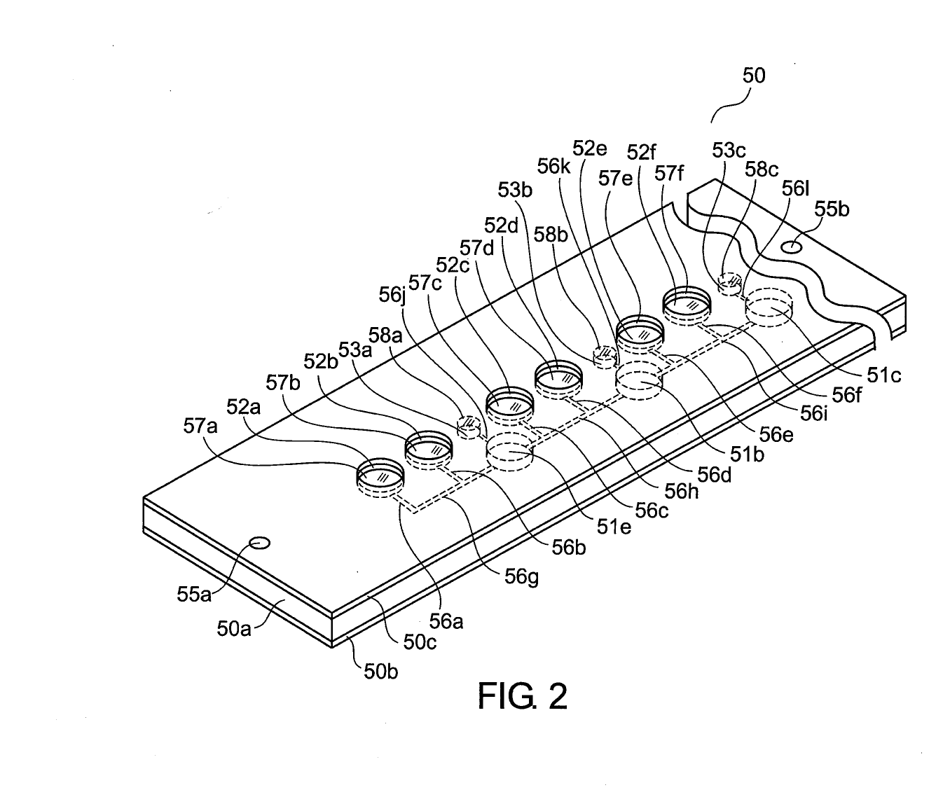

FIG. 2 is a perspective view showing details of the microchip 50.

The microchip 50 is of a three-layer structure consisting of a main plate 50a, a lower plate 50b, and an upper plate 50c, and has specimen vessels 52a, 52b, 52c, 52d, 52e, 52f passing through the main plate 50a and upper plate 50c and each having a container shape. The microchip 50 further has reaction vessels 51a, 51b, 51c passing through the main plate 50a and each having a container hole shape sealed with the lower plate 50b and upper plate 50c, and discharging ports 53a, 53b, 53c passing through the main plate 50a and lower plate 50b. The specimen vessels 52a, 52b are linked to the reaction vessel 51a through micro-channels 56a, 56b, 56g formed on the side of the main plate 50a facing the lower plate 50b. The discharging port 53a and the reaction vessel 51a are linked to each other through a micro-channel 56j formed on the side of the main plate 50a facing the upper plate 50c. Further, filters 58a, 58b, 58c are provided at the upper ends of the discharging ports 53a, 53b, 53c such that circulating liquid passes through the filters.

Further, the reaction vessels 51a, 51b and the specimen vessels 52c, 52d are mutually linked through channels 56h, 56c, 56d on the side of the main plate 50a facing the lower plate 50b, while the discharging port 53b and the reaction vessel 51b are linked to each other through a channel 56k on the side of the main plate 50a facing the upper plate 50c.

Further, the reaction vessels 51b, 51c are linked to the specimen vessels 52e, 52f through channels 56i, 56e, 56f on the side of the main plate 50a facing the lower plate 50b, while the discharging port 53c and the reaction vessel 51c are connected to each other through a channel 56l on the side of the main plate 50a facing the upper plate 50c.

On the other hand, pin holes 55a, 55b are provided in the end faces of the microchip 50 to pass through the main plate 50a, the lower plate 50b and the upper plate 50c and to function as guide means when the microchip 50 is mounted.

The specimen vessels 52a, 52b, 52c, 52d, 52e, 52f are preliminarily filed with predetermined volumes of specimens 57a, 57b, 57c, 57d, 57e, 57f. In general, the specimen 57a is a sample liquid containing chemical specimens such as genes to be analyzed, and the specimens 57b, 57c, 57d, 57e, 57f are specimen liquids to be sequentially reacted with the sample specimen 57a to extract specific genes. The specimens 52a, 52b, 52c, 52d, 52e, 52f will not leak out since they are transferred to the micro-channels 56a, 56b, 56c, 56d, 56e, 56f which are fine enough to prevent the specimens from flowing out by surface tension.

Next, operation of the first embodiment of this invention will be described with reference to FIGS. 1 to 11 and FIG. 17.

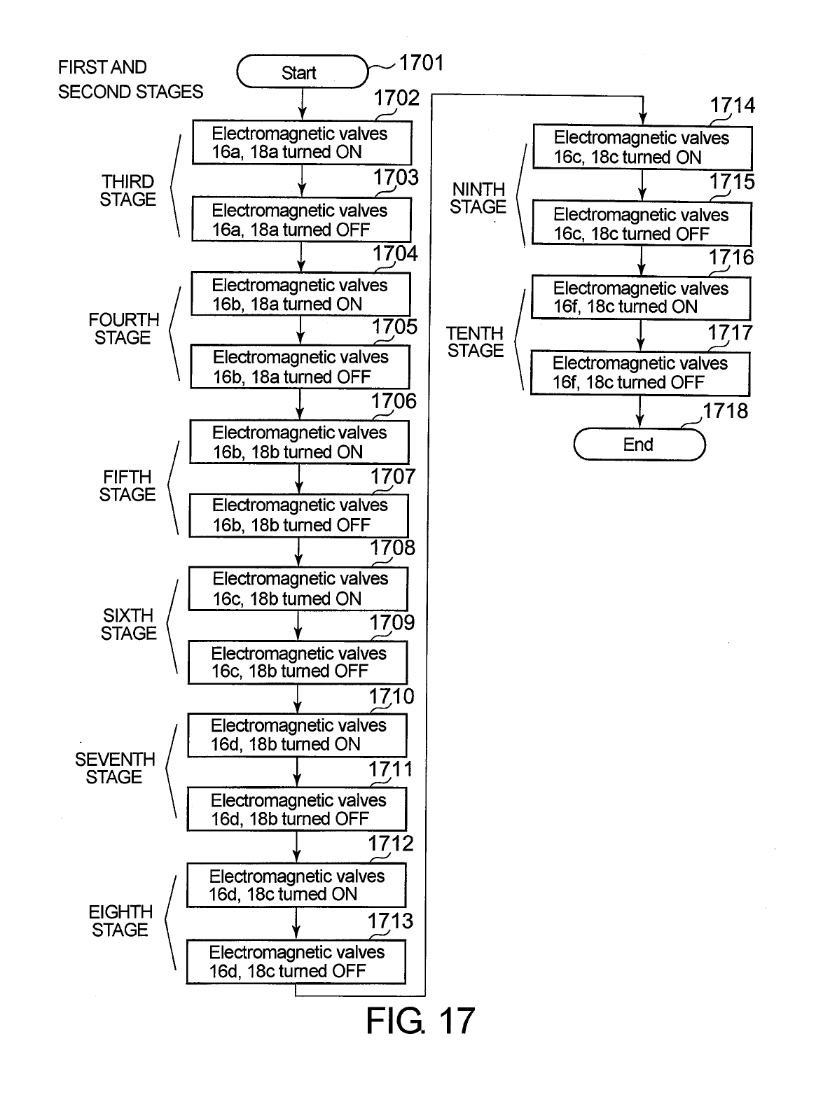

The first stage of operation is shown in FIG. 1 (step 1701 in FIG. 17).

The microchip 50 is mounted on the table 3 by inserting the pins 10a, 10b into the pin holes 55a, 55b. The cover 20 is turned in the direction B so that the fastening screw 25 is engaged in and fastened to the threaded hole 4. When the microchip 50 is mounted on the table 3, the specimen vessels 52a, 52b, 52c, 52d, 52e, 52f on the microchip 50 and the pressurizing holes 22a, 22b, 22c, 22d, 22e, 22f in the cover 20 are both sealed with the O-rings 26 and located at positions corresponding to each other. The discharging ports 53a, 53b, 53c, 53d, 53e, 53f are sealed on the table 3 by the O-rings 6a, 6b, and 6c and fixed at the positions corresponding to those of the discharging holes 5a, 5b, and 5c.

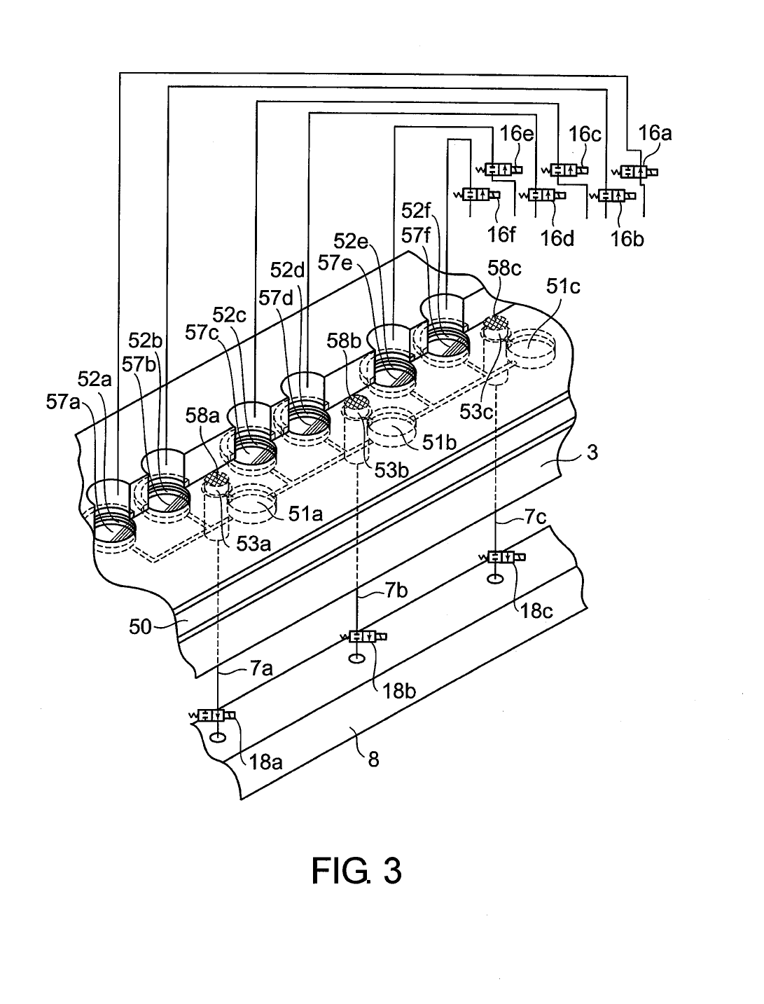

The second stage of the operation is shown in FIG. 3 (step 1701 in FIG. 17).

FIG. 3 shows an initial state in which the microchip 50 is mounted on the table 3. The pressurizing electromagnetic valves 16a, 16b, 16c, 16d, 16e, 16f are in deenergized state to turn off the pressure in the accumulator 11 shown in FIG. 1. The discharging electromagnetic valves 18a, 18b, 18c are also in deenergized state to turn off the tubes 7a, 7b, 7c extending from the discharging ports 53a, 53b, 53c to the waste vessel 8. The specimen vessels 52a, 52b, 52c, 52d, 52e, 52f are filled with the specimens 57a, 57b, 57c, 57d, 57e, 57d, while the reaction vessels 51a, 51b, 51c are vacant.

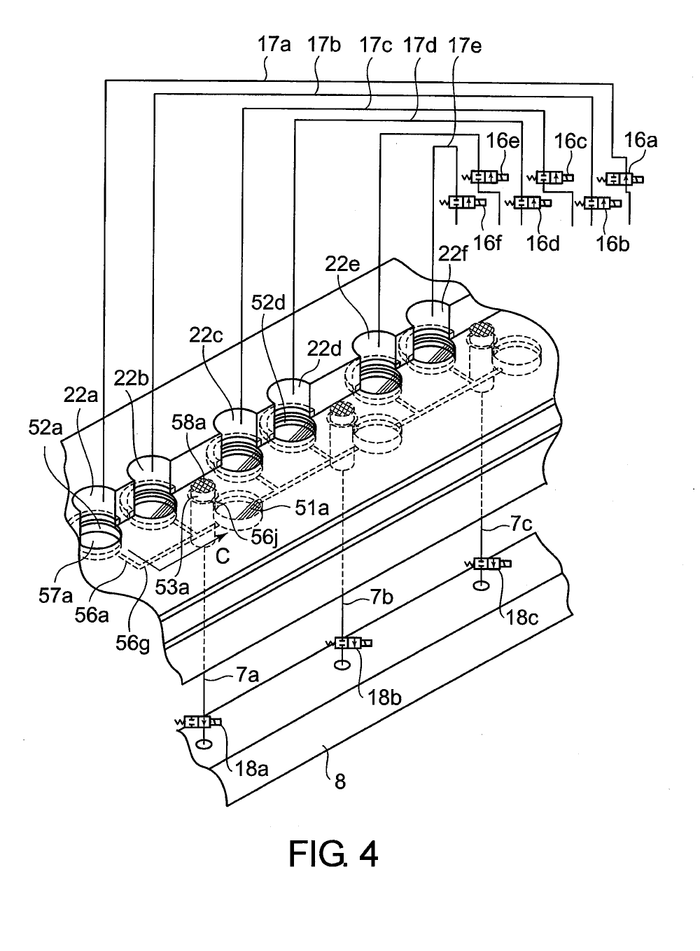

The third stage of the operation is shown in FIG. 4 (steps 1702 and 1703 in FIG. 17).

When the pressurizing electromagnetic valve 16a and the discharging electromagnetic valve 18a are energized, the pressure in the accumulator 11 shown in FIG. 1 is introduced into the pressurizing hole 22a via the pressurizing electromagnetic valve 16a and the tube 17a. On the other hand, as for the pressurizing holes 22b, 22c, 22d, 22e, 22f, since the pressurizing electromagnetic valve 16b, 16c, 16d, 16e, 16f are deenergized, the connecting tubes 17b, 17c, 17d, 17e, 17f are turned off. Further, the discharging electromagnetic valves 18b, 18c are deenergized and thus the connecting tubes 7b, 7c including in the same circuits as the discharging electromagnetic valves 18b, 18c are turned off. Since the tube 7a is the only path open to the waste vessel 8, the specimen 57a in the specimen vessel 52a is introduced into the waste vessel 8 through the channels 56a, 56g and via the reaction vessel 51a, the discharging hole 53a, the filter 58a, the tube 7a, and the discharging electromagnetic valve 18a. When this is done, the channels 56a, 56g are located under the reaction vessel 52a. The channel 56j functions as an outlet from above the reaction vessel 51a. After introducing the specimen 57a into the reaction vessel 52a, the channel 56j introduces only pressurized gas to the waste vessel 8 via the channel 56j, the discharging hole 53a, the filter 58a, the tube 7a, and the discharging electromagnetic valve 18a, while leaving the specimen 52a in the reaction vessel 51a due to passing resistance occurring at the filter 58a. This means that the specimen 57a contained in the specimen vessel 52a is transferred in a direction C to the reaction vessel 51a. After that, the pressurizing electromagnetic valve 16a and the discharging electromagnetic valve 18a are deenergized by a preinstalled program controlled by the controller 15 shown in FIG. 1, whereby the relevant circuit is turned off.

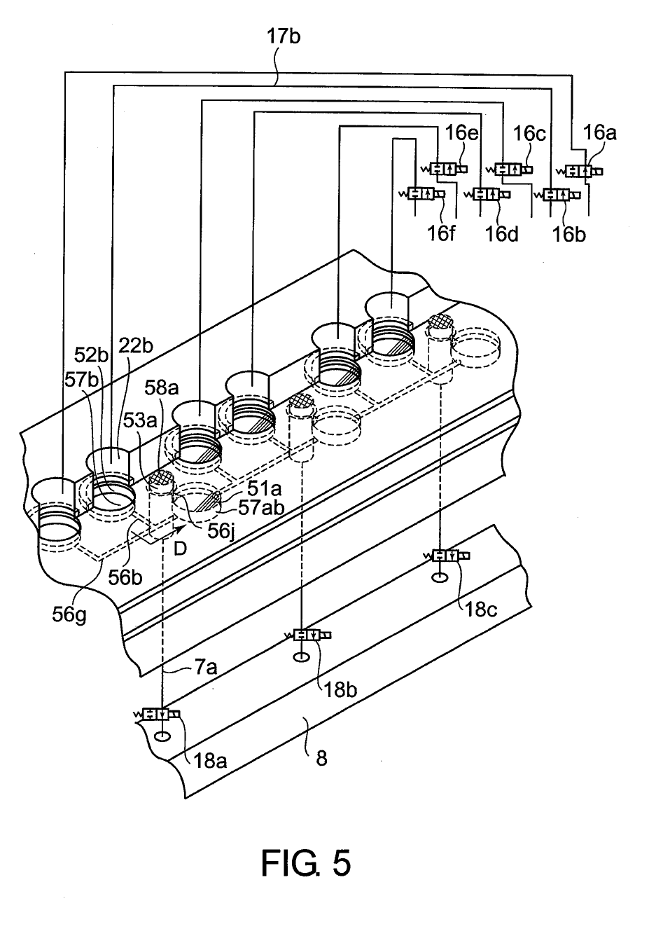

The fourth stage of the operation is shown in FIG. 5 (steps 1704 and 1705 in FIG. 17).

Once the pressurizing electromagnetic valve 16b and the discharging electromagnetic valve 18a are energized by a signal from the controller 15 shown in FIG. 1, the pressurized gas is introduced into the reaction vessel 52b via the pressurizing electromagnetic valve 16b, the tube 17b and the pressurizing hole 22b to push out the specimen 57b. Since the pressurizing electromagnetic valves 16a, 16c, 16d, 16e, 16f and the discharging electromagnetic valves 18b, 18c are closed, the specimen 57b flows out into the waste vessel 8, passing through the sole open path, namely through the channels 56b, 56g, the reaction vessel 51a, the channel 56j, the discharging port 53a, the filter 58a, the tube 7a, and the discharging electromagnetic valve 18a, like the operation as described above. However, since the reaction vessel 51a has already been filled with the specimen 57a transferred by the previous operation described above, the specimen 57a is mixed with the newly transferred specimen 57b to form a specimen mixture 57ab. A volume of the specimen mixture 57ab exceeding the capacity of the reaction vessel 51a and compressed gas additionally supplied are fed in a direction D to be discharged into the waste vessel 8 via the channel 56j, the discharging port 53a, the filter 58a, the tube 7a, and the discharging electromagnetic valve 18a. The pressurizing electromagnetic valve 16b and the discharging electromagnetic valve 18a are then deenergized by the preinstalled program, whereby the relevant circuit is blocked. As a result, the reaction vessel 51a is filled with the specimen mixture 57ab and the specimens react with each other.

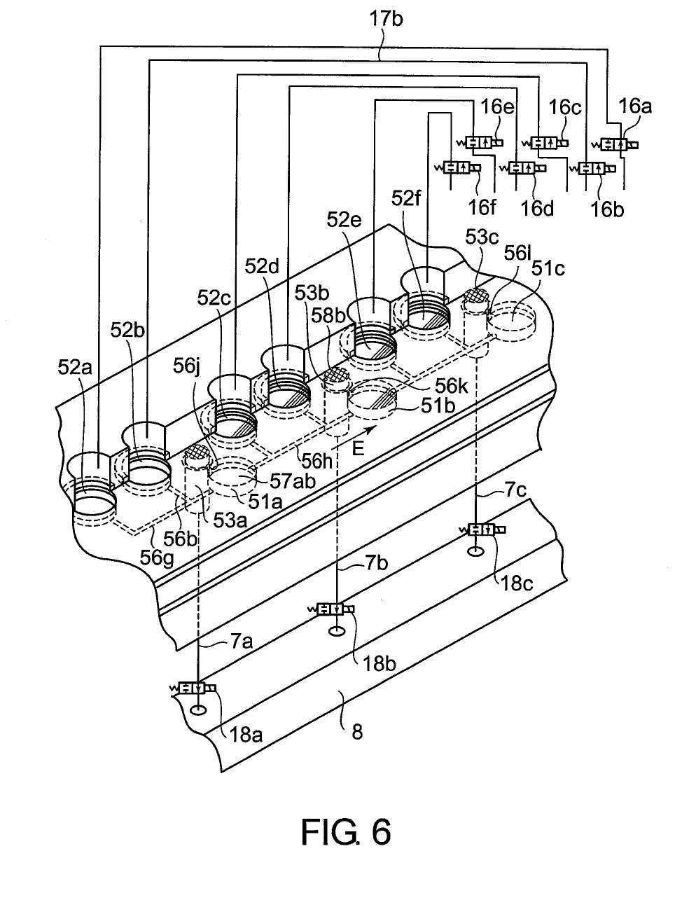

The fifth stage of the operation is shown in FIG. 6 (steps 1706 and 1707 in FIG. 17).

Once the pressurizing electromagnetic valve 16b and the discharging electromagnetic valve 18b are then energized by the preinstalled program, the specimen vessel 52b is pressurized via the pressurizing electromagnetic valve 16b and the tube 17b. Since the pressurizing electromagnetic valve 16a for the specimen vessel 52a is closed, the pressurized gas is fed to the reaction vessel 51a through the channels 56b, 56g. On the other hand, since the discharging electromagnetic valve 18a is closed, the channel 56j, the discharging port 53a, and the tube 7a form a closed circuit. Therefore, the pressurized gas introduced into the reaction vessel 51a stays in the upper part of the reaction vessel 51a and pressurizes the specimen mixture 57ab previously present in the reaction vessel 51a. The specimen vessel 52c and the specimen layer 52d are closed by the closed pressurizing electromagnetic valves 16c, 16d, and the specimen vessels 52e, 52f located upstream of the reaction vessel 51b are also closed by the closed pressurizing electromagnetic valves 16e, 16f. Further, the channel 56l, the discharging port 53c, and the tube 7c for the reaction vessel 51c are closed by the closed discharging electromagnetic valve 18c. As a result, the specimen mixture 57ab in the reaction vessel 51a is fed in a direction E. Specifically, the specimen mixture 57ab is fed into the waste vessel 8 by way of the discharging electromagnetic valve 18b, that is the only valve opened, via the channel 56h, the reaction vessel 51b, the channel 56k, the discharging port 53b, the filter 58b, and the tube 7b. Further, the specimen mixture 57ab fed to the reaction vessel 51b is flows into the reaction vessel 51b from below. Since the channel 56k is located above the reaction vessel 51b and passing resistance occurs at the filter 58b, the specimen mixture 57ab stays in the reaction vessel 51b, while only the pressurized gas is discharged into the waste vessel 8 through the channel 56k, the discharging port 53b, the filter 58b, the tube 7b, and the discharging electromagnetic valve 18b. As a result, the specimen mixture 57ab contained in the reaction vessel 51a is transferred to the reaction vessel 51b. The pressurizing electromagnetic valve 16b and the discharging electromagnetic valve 18b are then deenergized by the preinstalled program.

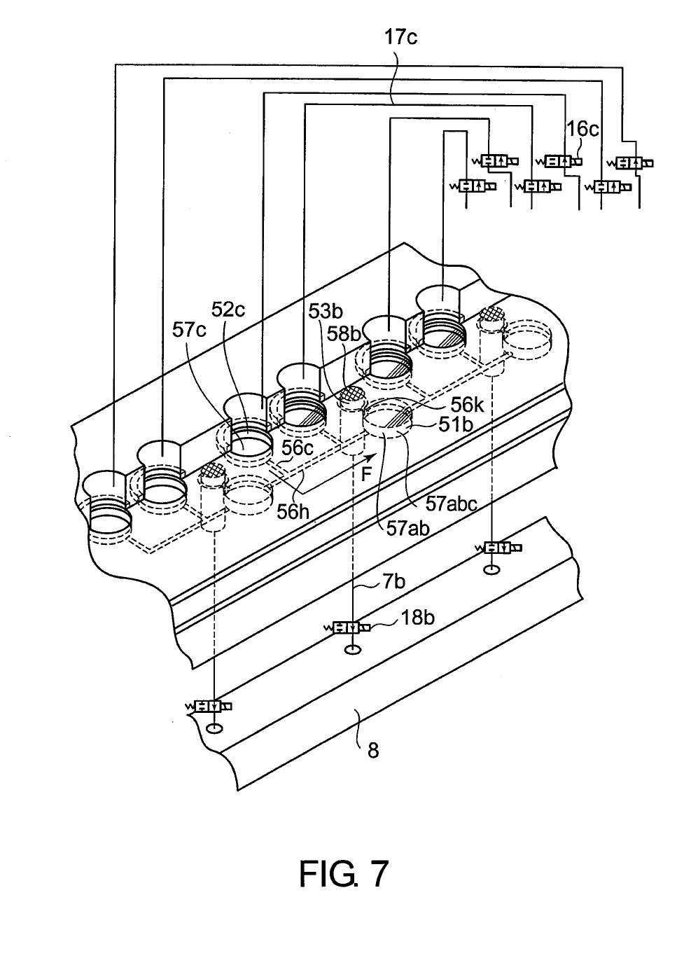

The sixth stage of the operation is shown in FIG. 7 (steps 1708 and 1709 in FIG. 17).

Once the pressurizing electromagnetic valve 16c and the discharging electromagnetic valve 18b are energized, the specimen 57c contained in the specimen vessel 52c is pressurized through the tube 17c, and fed to the only path opened to a direction F, namely through the channels 56c, 56h, the reaction vessel 51b, the channel 56k, the discharging port 53b, the filter 58b, the tube 7b, the discharging electromagnetic valve 18b, and introduced into the waste vessel 8. The specimen 57c flows, through the channel 56h, into the reaction layer 51b that has already been filled with the specimen mixture 57ab. Since the channel 56k to which the specimen flows out is disposed above the reaction vessel 51b, the specimen 57c is further mixed with the specimen mixture 57ab contained in the reaction vessel 52b to produce a specimen mixture 57abc, and the overflowing specimen mixture 57abc is discharged, together with compressed gas further supplied, into the waste vessel 8 via the channel 56k, the discharging port 53b, the filter 58b, the tube 7b, and the discharging electromagnetic valve 18b. As a result, some of the specimen mixture 57abc remains in the reaction vessel 51b. The pressurizing electromagnetic valve 16c and the discharging electromagnetic valve 18b are then deenergized by the preinstalled program.

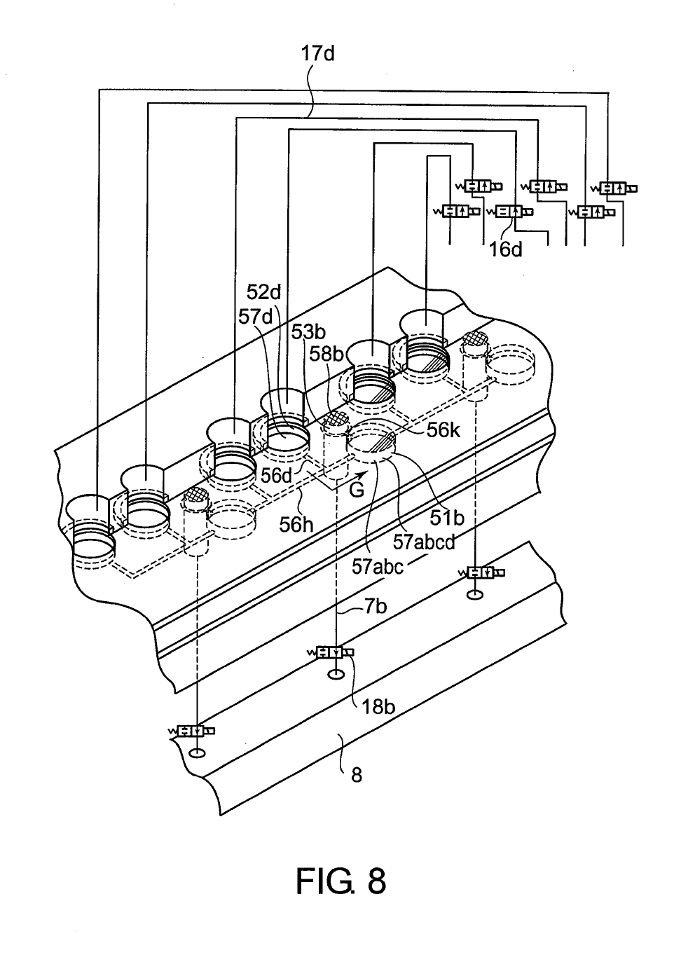

The seventh stage of the operation is shown in FIG. 8 (steps 1710 and 1711 in FIG. 17).

Once the pressurizing electromagnetic valve 16d and the discharging electromagnetic valve 18b are energized, the specimen 57d contained in the specimen vessel 52d is pressurized through the tube 17d, and is fed to the only path opened to a direction G, specifically through the channels 56d, 56h, the reaction vessel 51b, the channel 56k, the discharging port 53b, the filter 58b, the tube 7b, the discharging electromagnetic valve 18b, and introduced into the waste vessel 8. The specimen 57d flows, through the channel 56d, into the reaction vessel 51b already filled with the specimen mixture 57abc, and a specimen mixture 57abcd is produced. Since the channel 56k is disposed above the reaction vessel 51b, the overflowing specimen mixture 57abcd and the compressed gas further supplied are discharged into the waste vessel 8 via the channel 56k, the discharging port 53b, the filter 58b, the tube 7b, and the discharging electromagnetic valve 18b. As a result, the specimen mixture 57abcd remains and is stored in the reaction vessel 51b. After that, the pressurizing electromagnetic valve 16b and the discharging electromagnetic valve 18b are deenergized by the preinstalled program.

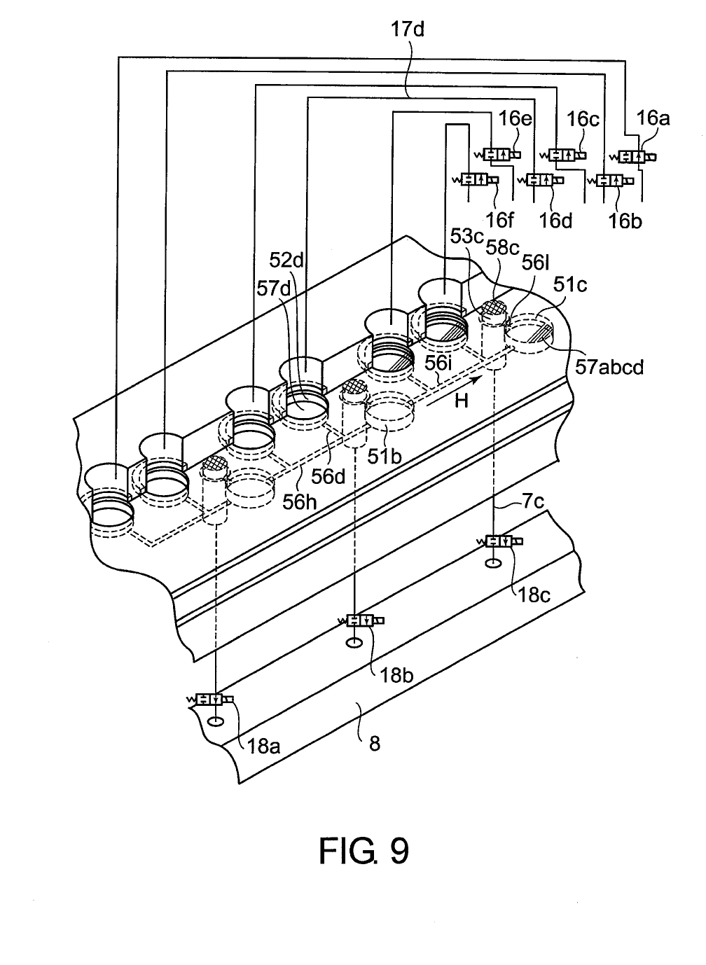

The eighth stage of the operation is shown in FIG. 9 (steps 1712 and 1713 in FIG. 17).

Once the pressurizing electromagnetic valve 16d and the discharging electromagnetic valve 18c are energized, the specimen vessel 52d to which the specimen 57d has already been transferred is pressurized through the pressurizing electromagnetic valve 16d and the tube 17d. Since the pressurizing electromagnetic valves 16a, 16b, 16d, 16e, 16f, and the discharging electromagnetic valve 18a, 18b are closed, the compressed gas pressurizing the specimen vessel 52d is fed to the only path opened to a direction H, namely through the channel 56d, the reaction vessel 51b, the channel 56i, the reaction vessel 51c, the channel 56l, the discharging port 53c, the filter 58c, the tube 7c, the discharging electromagnetic valve 18c, and into the waste vessel 8. While the reaction vessel 51b has already been filled with the specimen mixture 57abcd, the compressed gas flowing into the reaction vessel 51b from the channel 56h is contained in the upper part of the reaction vessel 51b to push out the specimen mixture 57abcd and feed the same to the channel 56i, and causes the specimen mixture 57abcd to flow into the reaction vessel 51c. Since the channel 56l serving as a discharge path is disposed above the reaction vessel 51c, and passing resistance occurs at the filter 58c, the compressed gas which has pushed out is fed into the waste vessel 8 through the channel 56l via the discharging hole 53c, the filter 58c, the tube 7c, the discharging electromagnetic valve 18c, while leaving the specimen mixture 57abcd in the reaction vessel 51c. As a result, the specimen mixture 57abcd contained in the reaction vessel 51b is transferred to the reaction vessel 51c. After that, the pressurizing electromagnetic valve 16d and the discharging electromagnetic valve 18c are deenergized by the preinstalled program.

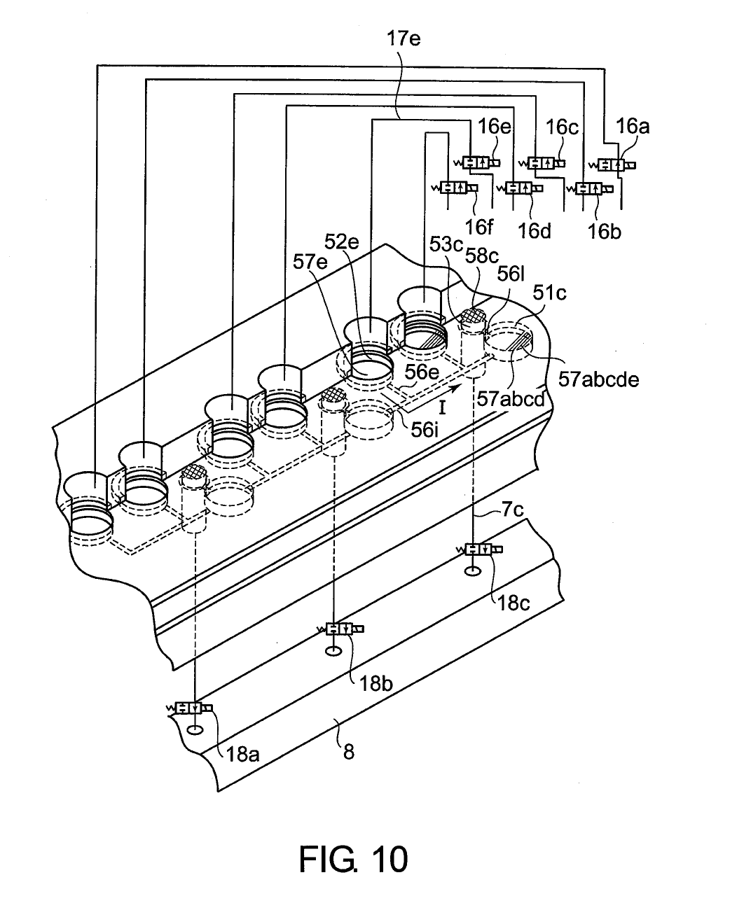

The ninth stage of the operation is shown in FIG. 10 (steps 1714 and 1715 in FIG. 17).

The pressurizing electromagnetic valve 16e and the discharging electromagnetic valve 18c are energized. When the specimen vessel 52e filled with the specimen 57e is pressurized through the pressurizing electromagnetic valve 16e and the tube 17e, the specimen 57e is fed to the only path opened to a direction I, namely through the channels 56e, 56i, the reaction vessel 51c, the channel 56l, the discharging port 53c, the filter 58c, the tube 7c, the discharging electromagnetic valve 18c and into the waste vessel 8, since the pressurizing electromagnetic valves 16a, 16b, 16c, 16d, 16f and the discharging electromagnetic valves 18a, 18b are closed. The specimen 52e thus pushed out flows into the reaction vessel 51c from the channel 56i linked to the lower side of the reaction vessel 51c which has already been filled with the specimen mixture 57abcd in the previous step, and reacts with the specimen mixture 57abcd to produce a specimen mixture 57abcde. Further, the overflowing specimen mixture 57abcde and the compressed gas further supplied are discharged from the channel 56l disposed above the reaction vessel 51c into the waste vessel 8 via the discharging port 53c, the filter 58c, the tube 7c, and the discharging electromagnetic valve 18c. As a result, the reaction vessel 51c is filled with the specimen mixture 57abcde. After that, the pressurizing electromagnetic valve 16e and the discharging electromagnetic valve 18c are deenergized.

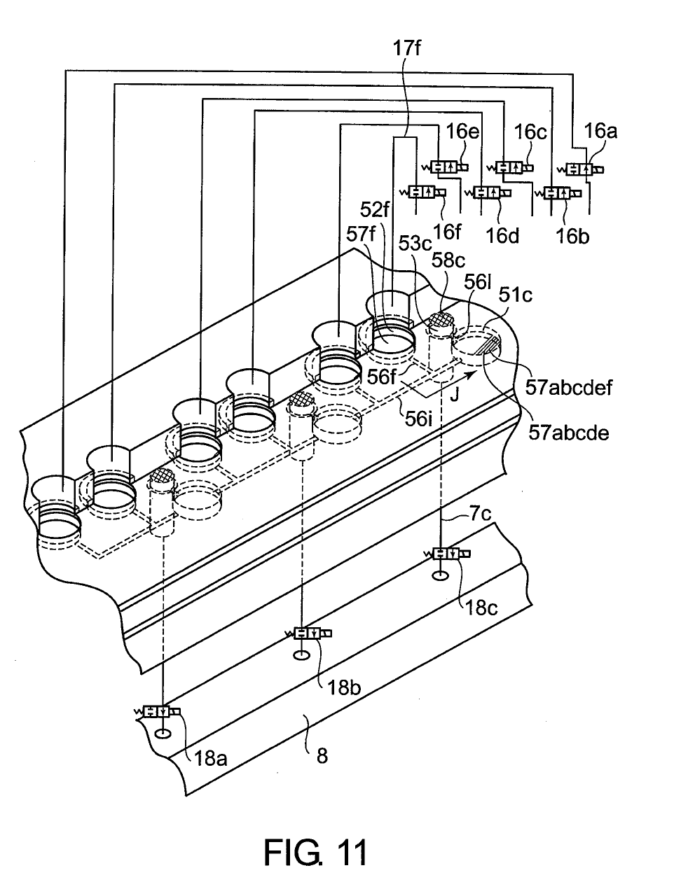

The tenth stage of the operation is shown in FIG. 11 (steps 1716 and 1717 in FIG. 17).

The pressurizing electromagnetic valve 16f and the discharging electromagnetic valve 18c are energized. When the specimen vessel 52f is pressurized through the pressurizing electromagnetic valve 16f and the tube 17f, the specimen 57f is fed to the only path opened to a direction J, namely through the channels 56f, 56i, the reaction vessel 51c, the channel 56l, the discharging port 53c, the filter 58c, the tube 7c, and the discharging electromagnetic valve 18c, and into the waste vessel 8, since the pressurizing electromagnetic valves 16a, 16b, 16c, 16d, 16e and the discharging electromagnetic valves 18a, 18b are closed. While the reaction vessel 51c has already been filled with the specimen mixture 57abcde in the previous step, the specimen 57f is additionally fed into the reaction vessel 51c from the channel 56i linked to the lower side of the reaction vessel 51c, and a specimen mixture 57abcdef is produced. The overflowing specimen mixture 57abcdef and the compressed gas further supplied are discharged from the channel 56l provided above the reaction vessel 51c into the waste vessel 8 via the discharging port 53c, the filter 58c, the tube 7c, and the discharging electromagnetic valve 18c. As a result, some specimen mixture 57abcdef is left and contained in the reaction vessel 51c. After that, the pressurizing electromagnetic valve 16f and the discharging electromagnetic valve 18c are deenergized.

As seen from the description above, the specimens 57a and 57b are mixed together and caused to react for a certain period of time in the reaction vessel 51a, and the mixture product is transferred to the reaction vessel 51b. The specimens 57c and 57d are additionally fed into the reaction vessel 51b where they are caused to react for a certain period of time, and then the product is transferred into the reaction vessel 51c. Further, the specimens 57e and 57f are added and caused to react, whereby the final product is obtained in the reaction vessel 51c, and a series of transfer processing steps is terminated (step 1718 in FIG. 17).

### Other Exemplary Embodiments of the Invention

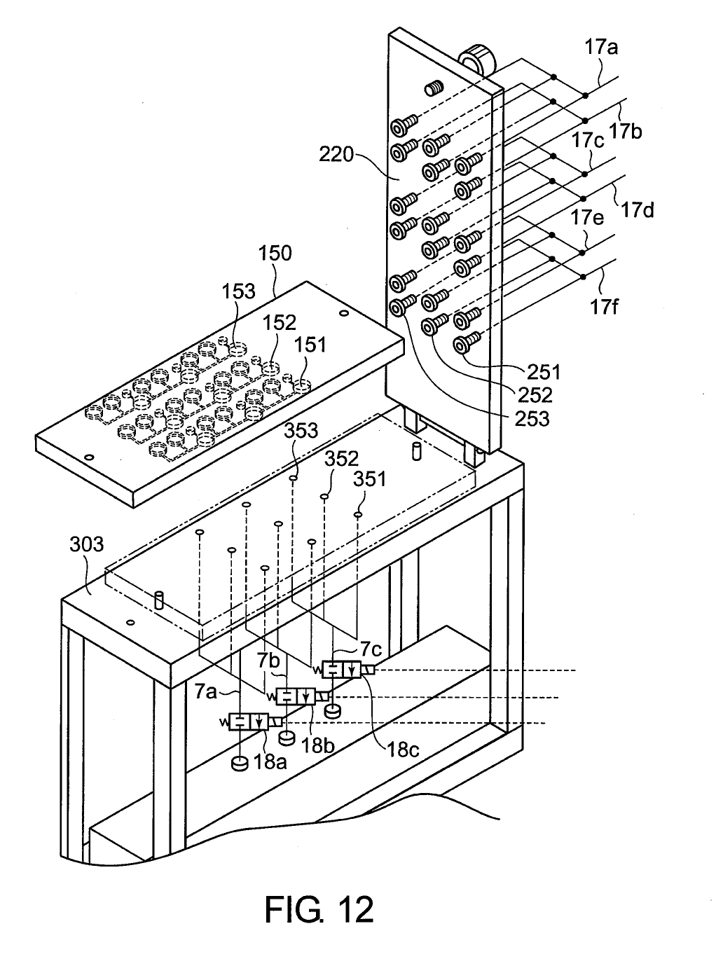

Another exemplary embodiment of this invention is shown in FIG. 12.

There is provided on a microchip 150 a reaction line 151 composed of the reaction vessels 51a, 51b, 51c, the specimen vessels 52a, 52b, 52c, 52d, 52e, 52f, the discharging holes 53a, 53b, 53c, and the channel 56 shown in FIG. 1. Further, reaction lines 152, 153 having a similar mechanism configuration to that of the reaction line 151 are additionally provided. A cover 220 is provided with pressurizing hole groups 251, 252, 253 each composed of the pressurizing holes 22a, 22b, 22c, 22d, 22e, 22f and the O-rings 26 shown in FIG. 1. Further, there are provided, on a table 303, discharging hole groups 351, 352, 353 each composed of the discharging holes 5a, 5b, 5c and the O-rings 6a, 6b, 6c shown in FIG. 1.

The pressurizing hole groups 251, 252, 253 on the cover 220 are engaged with paths branched from the tubes 17a, 17b, 17c, 17d, 17e, 17f in the same manner as the paths shown in FIG. 1. The tubes 7a, 7b, 7c connected to and extending from the discharging electromagnetic valves 18a, 18b, 18c are branched and connected to the discharging hole groups 351, 352, 353 in the same manner as shown in FIG. 1. It is made possible, by providing the configuration described above and transferring each of the specimens as described above, to drive a plurality of reaction lines 151, 152, 153 simultaneously. Furthermore, this embodiment provides an advantage that a greater number of reaction processes can be performed at the same time since the discharging electromagnetic valves 18a, 18b, 18c and the pressurizing electromagnetic valves 16a, 16b, 16c, 16d, 16e, 16f shown in FIG. 1, which serve as drive means, can be used in common. Although the description above has been made in terms of a case in which there are three reaction lines, an equivalent result can be obtained even if more reaction lines are provided.

While the description has been made above on the first to tenth stages of the operation, it is obvious that the same result can be obtained eve if the filters 58a, 58b, 58c provided on the discharging channels are omitted depending on the characteristics (such as viscosity) of the specimens 57a, 57b, 57c, 57d, 57e, 57f.

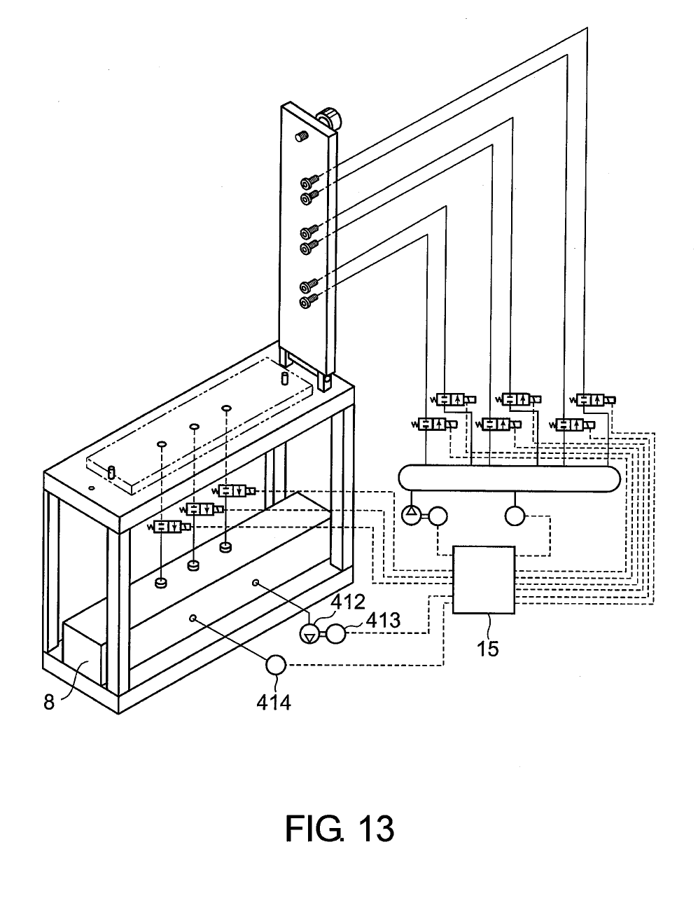

Still another embodiment of this invention is shown in FIG. 13.

The waste vessel 8 has a sealed structure, and is provided with a negative pressure pump 412 for establishing a negative pressure in the inside of the waste vessel 8, and with a drive motor 413. Further, a pressure sensor 414 is further connected to the waste vessel 8 for detecting and feeding back the pressure in the waste vessel 8. The motor 413 and the pressure sensor 414 are connected to a controller 15 so that the pressure in the waste vessel 8 is controlled to a predetermined negative value. The configuration as described above makes it possible that specimens and compressed gas can be discharged into the waste vessel 8 more reliably than when the inside of the waste vessel 8 is maintained at the atmospheric pressure, and thus the time required for discharging is shortened and the productivity is improved.

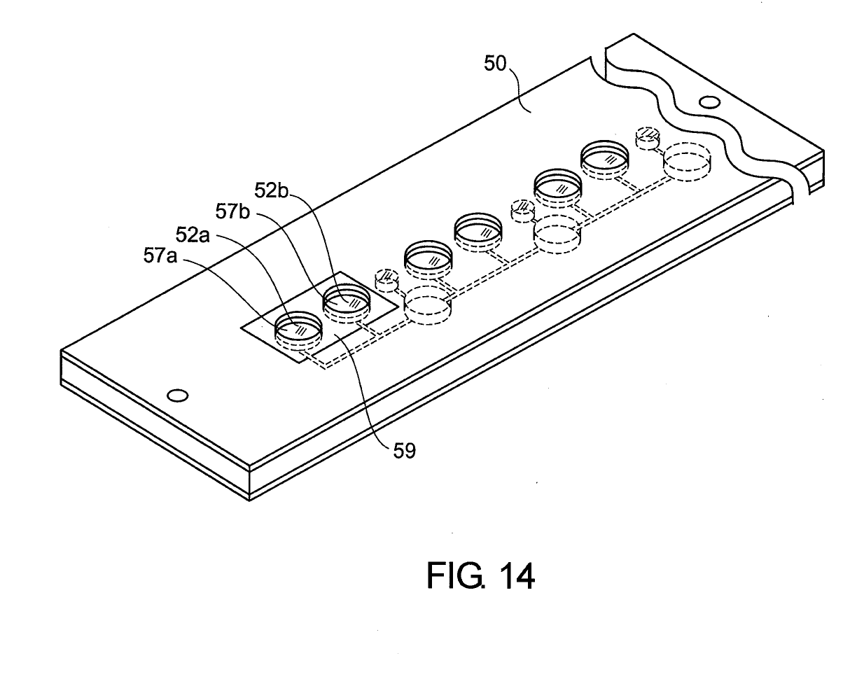

Still another embodiment of this invention is shown in FIG. 14,

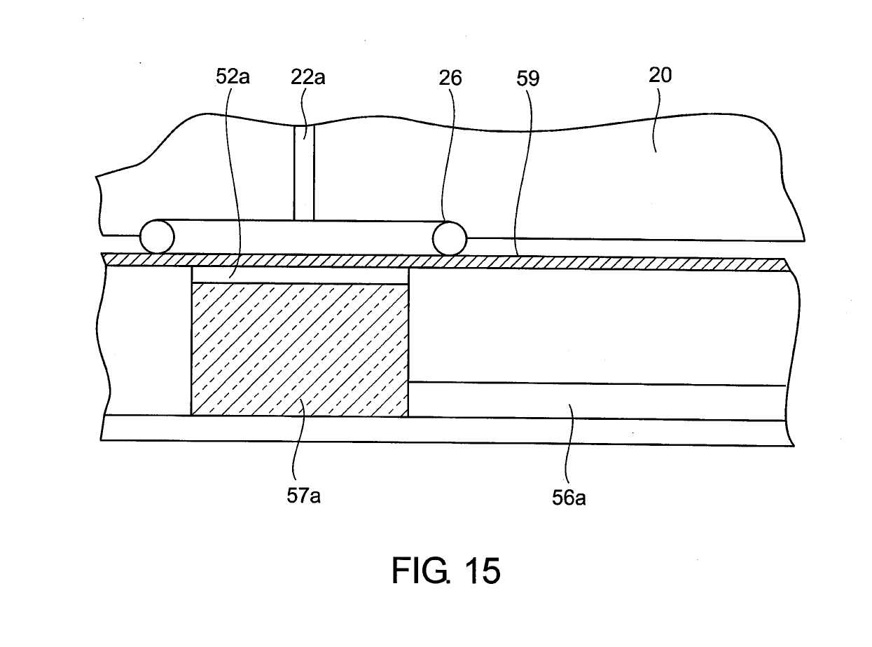

Specimen vessels 52a, 52b provided in a microchip 50 are filled with specimens 57a, 57b, and a stretchable film 59 is disposed on the top of the specimen vessels. FIG. 15 is a cross-sectional view showing a configuration including the specimen 57a contained in the specimen vessel 52a, the cover 20, the pressurizing hole 22a, the O-ring 26, the channel 56a, and the film 59 described above.

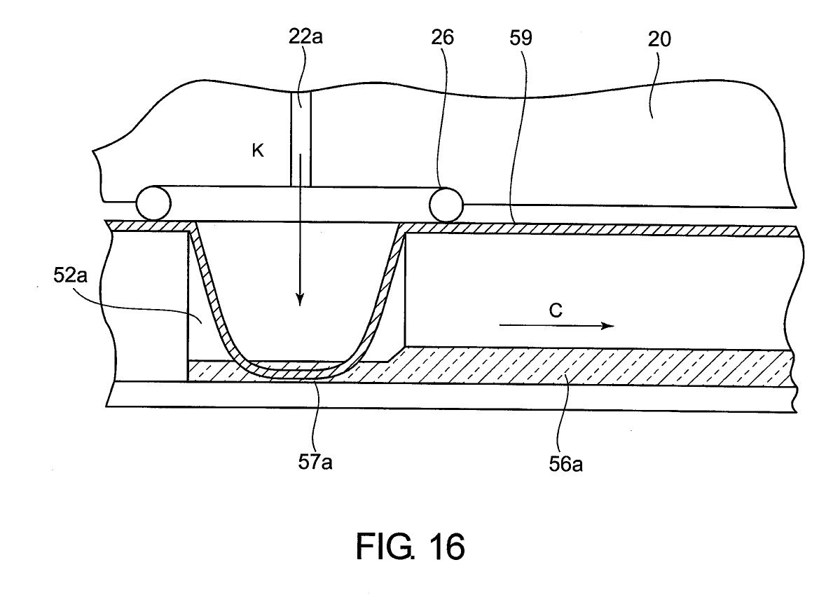

Operation of this embodiment will be described with reference to FIG. 16.

Since the film 59 is sealed with the O-ring 26, compressed gas supplied through the pressurizing hole 22a formed in the cover 20 causes the film 50 to bulge downward in the specimen vessel 52a. The specimen 57a in the specimen vessel 52a is thereby pressurized and pushed out to the direction of the channel 56a. This makes it possible to prevent supply of excessive gas and to improve the accuracy in quantity of transferred specimens without the need of using an expensive micro pump having high flow control accuracy. The quantity of transferred specimens can be controlled by changing the combination of the size of the specimen vessel 52a, the material of the film 59, and the pressure of the compressed gas to be supplied.

When this device is operated in the atmospheric air or the like, gas such as air will exist around the pressurizing hole 22a formed in the cover 20 if the specimen vessel 52a in the microchip 50 is filled with a specimen, the stretchable film 59 is disposed thereon, and then the cover 20 is placed to cover the same. However, since compressed gas is supplied through the pressurizing hole 22a formed in the cover 20, the air (gas) existing around the pressurizing hole 22a will not be mixed in and will not pose any problem. This detachable construction of the cover makes it possible to replace the microchip 50 for every analysis, and to prevent the contamination possibly caused by mixture of specimens to be tested. As a result, the device can be simplified in configuration, while the fault tolerance and reliability can be improved.

Since the cover 20 is detachable, as described above, the stretchable film 59 to be disposed on the top of the specimen vessel 52a in the microchip 50 also can be made detachable. This makes it possible to introduce a specimen into the specimen vessel 52a from the top of the microchip 50. In addition, since the channel 56a is disposed under the specimen vessel 52a, part of the specimen introduced into the lower part of the specimen vessel 52a is first pushed out into the channel 56a, even if the specimen has not been introduced completely into the specimen vessel 52a and some gas is brought into the upper part of the specimen vessel 52a. It is made possibly, by changing the combination of the size of the specimen vessel 52a, the material of the film 59, and the pressure of the compressed gas to be supplied, to transfer only the specimen while leaving the gas, which might be mixed into the specimen, in the upper part of the specimen vessel 52a. As a result, the handling of the device is simplified and the fault tolerance is improved.

Using a transfer mechanism according to an aspect of this invention, a plurality of chemical specimens can be sequentially transferred to a plurality of reaction vessels in a microchip by simple configuration and control, so that they can be reacted to efficiently produce a product required for gene analysis. Further, the size reduction of the device makes it possible to reduce the weight, to increase the operating speed, and reduce the power consumption.

Specimens according to an aspect of the invention may be any substance of any form as long as it can be transferred by the transfer mechanism. Specifically, the chemical specimens that can be transferred in the microchip may assume the form of liquid, gas, gel, powder, or the like. Taking this function into consideration, it will be understood that this invention is applicable to analysis of gas containing bacteria or the like.

Further, using this microchip transfer mechanism, the need is eliminated of providing drive means for the transfer purpose within the microchip, and it is made possible to provide disposable, inexpensive and small-sized microchips. Thus, the washing process conventionally required before every reuse of the microchip can be omitted, making it possible to perform gene analyses at a low cost and yet to improve the analysis reliability.

Further, this microchip transfer mechanism allows a large number of reaction lines to operate simultaneously with the use of a single drive means for the transfer purpose, resulting in significant improvement in working efficiency, in reliability, and in operability.

As described above, this invention provides a microchip fluid control mechanism having a plurality of specimen vessels each having an open top for receiving specimens, and a plurality of reaction vessels for mixing and reacting the specimens, the specimen vessels and the reaction vessels being mutually linked through channels so that the specimens are sequentially transferred with the use of pressurizing means and subjected to predetermined processing. The microchip fluid control mechanism of the invention is characterized in that transfer channels extending from the specimen vessels and transfer channels to the reaction vessels are provided under the specimen vessels and the reaction vessels.

The predetermined processing is processing to react, mix, separate, or analyze the specimens, or processing to extract, react, or analyze genes.

Preferably, compressed gas is supplied under pressure by the pressurizing means from an opening provided at the top of each of the specimen vessels, so that the specimens are transferred, together with the compressed gas, to the reaction vessels.

Preferably, the transfer channels extending from the reaction vessels are provided on and above the reaction vessels while the transfer channels are opened to below the microchip.

When the transfer channel extending from each of the specimen vessels and the transfer channel extending to each the reaction vessels are formed as a single reaction line, it is preferred that the reaction line be provided in plurality on the microchip, and a single pressurizing means be branched to drive the plurality of reaction lines.

Preferably, the microchip transfer mechanism further has negative pressure generation means and a discharging vessel for collecting the pressurized gas and the specimens discharged thereto, and a negative pressure is established in the inside of the discharging vessel by driving the transfer channels extending from the reaction vessels by the negative pressure generation means.

It is also preferred that a filter be provided in each of the transfer channels extending from the reaction vessels so as to leave the specimens in the reaction vessels.

Preferably, a stretchable film is provided on the top face of each specimen vessel, and when the specimen is transferred, the specimen vessel is pressurized through the stretchable film to feed out the specimen. The stretchable film is preferably provided detachable.

Further, this invention provides a microchip fluid control mechanism having a plurality of specimen vessels each having an open top for receiving specimens, and a plurality of reaction vessels for mixing and reacting the specimens, the specimen vessels and the reaction vessels being mutually linked through channels so that the specimens are sequentially transferred with the use of pressurizing means and subjected to predetermined processing, and characterized in that: the specimens are transferred by supplying compressed gas from above the specimen vessels; transfer channels to the reaction vessels are provided under the microchip, while transfer channels extending from the reaction vessels are provided on the microchip; and pressurizing means for supplying compressed gas from a member holding the microchip is provided outside the microchip.

The predetermined processing is processing to react, mix, or analyze the specimens, or processing to extract, react, or analyze genes.

Further, this invention provides a microchip fluid control mechanism having a plurality of specimen vessels each having an open top for receiving specimens, and a plurality of reaction vessels for mixing and reacting the specimens, the specimen vessels and the reaction vessels being mutually linked through channels so that the specimens are sequentially transferred with the use of pressurizing means and subjected to predetermined processing, the microchip fluid control mechanism being characterized in that: the microchip is composed of a lower plate, an upper plate, and a main plate interposed between the lower plate and the upper plate; the specimen vessels have a container shape passing through the main plate and the upper plate; the reaction vessels have a container hole shape passing through the main plate and sealed with the lower plate and the upper plate; a plurality of discharging ports are formed to pass through the main plate and the lower plate; the specimen vessels and the reaction vessels are linked to each other through a first channel provided on the side of the main plate facing the lower plate; and the discharging ports and the reaction vessels are linked to each other through a second channel provided on the side of the main plate facing the upper plate.

The predetermined processing is processing to react, mix, separate, or analyze the specimens, or processing to extract, react, or analyze genes.

It is preferred that the pressurizing means be provided outside the microchip.

Furthermore, according to another preferred aspect of the invention, there is provided, on the bottom side of a microchip with respect to a thickness direction thereof, a channel for discharging from a plurality of specimen container holes and for injecting into specimen reaction container holes, and there is further provided, near the top face of the microchip, a channel for discharging specimens overflowing from a plurality of specimen reaction containers which specimens are injected into. This configuration allows a predetermined volume of specimens to remain in the specimen reaction containers.

According to another preferred aspect of the invention, the tops of the specimen container holes provided in the microchip are opened, and a presser cover for holding the microchip from above is provided with compressed gas applying holes at positions corresponding to the opened specimen containers, so that the specimens contained in the specimen containers are pushed out by the compressed gas.

According to another preferred aspect of the invention, in order to prevent a necessary volume of specimens from being discharged when the specimens, which have been transferred from a plurality of specimen containers and supplied to reaction vessels, overflow from the reaction vessels, a discharging channel port is provided in each of reaction vessel to be oriented downward from the top, while discharging channels are provided in a table for holding the microchip in cooperation with the cover at positions corresponding to the discharging channel ports, so that a necessary volume of the specimen pushed by the compressed gas remains in the reaction vessel while only excessive specimen is discharged.

According to another preferred aspect of the invention, a single transfer drive means is branched to drive a plurality of pairs of specimen reaction channels at the same time for improving the productivity.

According to another preferred aspect of the invention, the discharging channel provided in the table is further provided with suction means for sucking the specimens with negative pressure in order to reliably isolate the overflowing specimens to be discharged from the microchip, and a single transfer drive means is branched to drive a plurality of pairs of specimen reaction channels at the same time in order to improve the productivity.

According to another preferred aspect of the invention, in order to fill the reaction vessels with specimens efficiently, a filter is provided on the channel extending out from each reaction vessel so as to generate a difference between gas-passing resistance and liquid-passing resistance.

According to another preferred aspect of the invention, in order to enable transfer of stable volumes of specimens and to prevent excessive supply of gas for certain specimens, a stretchable film is provided on the top of each specimen vessel, and the specimen vessel is pressurized through the film to transfer the specimen by variation in capacity of the specimen vessel caused by bulging of the film.

According to this invention, disposable and inexpensive microchips can be provided by omitting valve mechanisms conventionally provided in microchips and thus simplifying the channel structure.

According to a preferred aspect of this invention, valve mechanisms conventionally provided in microchips are omitted and, instead, specimens are transferred by compressed gas supplied from a member holding a microchip. Therefore, this invention can provide disposable and inexpensive microchips.

The use of compressible medium (gas) here provides advantageous effects as described below. The device is surrounded by abundant air (gas). In the case of using incompressible medium (see Patent Document 3), care is required to prevent bubbles (of gas such as air) from being mixed into the incompressible medium. In order to prevent this, several measures must be taken. In contrast, in the case of using compressible medium (gas) as in this invention, operation is possible even if air (gas) in the surroundings is mixed into when the air (gas) is supplied from pressurizing holes. This consequently allows simplification of the device and improves the fault tolerance thereof.

According a preferred aspect of this invention, the size of the device can be reduced and discharged specimens can be collected reliably. Thus, expensive specimens can be analyzed by using a minimum volume. Furthermore, when performing analyses repeatedly, it is possible to effectively prevent cross contamination with a specimen used in previous analysis.

According a preferred aspect of this invention, it is possible to drive a plurality of pairs of specimen reaction channels by using simple transfer drive means. This makes it possible to realize transfer with improved productivity with the use of an inexpensive and miniaturized mechanism.

According a preferred aspect of this invention, specimens discharged after use can be collected completely, whereby it is made possible, when analyses are performed repeatedly, to prevent cross contamination with specimens used in previous analyses.

According a preferred aspect of this invention, a plurality of pairs of specimen reaction channels can be driven simultaneously by using simple transfer drive means, and thus transfer with improved productivity can be realized by using an inexpensive and miniaturized mechanism.

According a preferred aspect of this invention, a stretchable film is provided on the top of a specimen vessel in a microchip, the specimen vessel being filled with a specimen, so that the specimen is transferred by being pressurized through the bulging film. This makes it possible to improve the accuracy of flow rate and to prevent the supply of excessive gas.

Although this invention has been described specifically based on its exemplary preferred embodiments, this invention is not limited to these embodiments. It will be understood that various modifications may be made without departing from the scope and spirit of the invention, and such modifications are obviously covered by the scope of this invention.

### INDUSTRIAL APPLICABILITY

According to this invention, specimens and liquid reagents are caused to react on a single chip. Therefore, the invention is applicable to medical diagnosis tools, bioresearch tools, food inspection systems, environmental inspection systems and so on by means of chemical refining, production and analysis, gene analysis, or cell proliferation.

This application is based upon and claims the benefit of priority from Japanese Patent Application No. 2007 54041, filed Mar. 5, 2007, the disclosure of which is incorporated herein in its entirety by reference.

1-17. (canceled)

18. A microchip fluid control mechanism, comprising:
a plurality of specimen vessels each having an open top for receiving specimens, and a plurality of reaction vessels for mixing and reacting the specimens, the specimen vessels and the reaction vessels being mutually through a channel to sequentially transfer the specimens by a pressurizing unit so that the specimens are subjected to predetermined processing, wherein:
transfer channels extending from the specimen vessels and transfer channels to the reaction vessels are provided under the specimen vessels and the reaction vessels.

19. The microchip fluid control mechanism as claimed in claim 18, wherein the predetermined processing is processing to react, mix, separate, or analyze the specimens.

20. The microchip fluid control mechanism as claimed in claim 18, wherein the predetermined processing is processing to extract, react, or analyze genes.

21. The microchip fluid control mechanism as claimed in claim 18, wherein compressed gas is supplied under pressure by the pressurizing unit from an opening provided at the top of each of the specimen vessels, so that the specimens are transferred, together with the compressed gas, to the reaction vessels.

22. The microchip fluid control mechanism as claimed in claim 18, wherein transfer channels extending from the reaction vessels are provided above the reaction vessels while the transfer channels are opened to below the microchip.

23. The microchip fluid control mechanism as claimed in claim 18, wherein when the transfer channel extending from each of the specimen vessels and the transfer channel extending to each of the reaction vessels are formed as a single reaction line, the reaction line is provided in plurality on the microchip, and a single pressurizing unit is branched to drive the plurality of reaction lines.

24. The microchip fluid control mechanism as claimed in claim 22, further comprising:
a negative pressure generation unit, and a discharging vessel for collecting the pressurized gas and the specimens discharged thereto, wherein a negative pressure is established in an inside of the discharging vessel by driving the transfer channels extending from the reaction vessels by the negative pressure generation unit.

25. The microchip fluid control mechanism as claimed in claim 22, wherein a filter is provided in each of the transfer channels extending from the reaction vessels so as to leave the specimens in the reaction vessels.

26. The microchip fluid control mechanism as claimed in claim 18, wherein a stretchable film is provided on a top surface of each of the specimen vessels, and when the specimen is transferred, the specimen vessel is pressurized through the stretchable film to feed out the specimen.

27. The microchip fluid control mechanism as claimed in claim 26, wherein the stretchable film is provided detachably.

28. A microchip fluid control mechanism, comprising:
a plurality of specimen vessels each having an open top for receiving specimens, and a plurality of reaction vessels for mixing and reacting the specimens, the specimen vessels and the reaction vessels being mutually linked through a channel to sequentially transfer the specimens so that the specimens are subjected to predetermined processing, wherein:
the specimens are transferred by supplying compressed gas from above the specimen vessels;
transfer channels to the reaction vessels are provided under the microchip, while transfer channels extending from the reaction vessels are provided on the microchip; and a pressurizing unit for supplying compressed gas from a member holding the microchip is provided outside the microchip.

29. The microchip fluid control mechanism as claimed in claimed 28, wherein the predetermined processing is processing to react, mix, or analyze the specimens.

30. The microchip fluid control mechanism as claimed in claimed 28, wherein the predetermined processing is processing to extract, react, or analyze genes.

31. A microchip fluid control mechanism, comprising:
a plurality of specimen vessels each having an open top for receiving specimens, and a plurality of reaction vessels for mixing and reacting the specimens, the specimen vessels and the reaction vessels being mutually linked through a channel to sequentially transfer the specimens by a pressurizing unit so that the specimens are subjected to predetermined processing, wherein:
the microchip is composed of a lower plate, an upper plate, and a main plate interposed between the lower plate and the upper plate;
the specimen vessels have a container shape passing through the main plate and the upper plate;
the reaction vessels have a container hole shape passing through the main plate and sealed with the lower plate and the upper plate;
a plurality of discharging ports are formed to pass through the main plate and the lower plate;
the specimen vessels and the reaction vessels are linked to each other through a first channel provided on a side of the main plate facing the lower plate; and
the discharging ports and the reaction vessels are linked to each other through a second channel provided on a side of the main plate facing the upper plate.

32. The microchip fluid control mechanism as claimed in claimed 31, wherein the predetermined processing is processing to react, mix, separate, or analyze the specimens.

33. The microchip fluid control mechanism as claimed in claimed 31, wherein the predetermined processing is processing to extract, react, or analyze genes.

34. The microchip fluid control mechanism as claimed in claimed 31, wherein the pressurizing unit is provided outside the microchip.
